(19) United States
(12) Patent Application Publication
(10) Pub. No.: US 2023/0233630 A1
(43) Pub. Date: Jul. 27, 2023

(54) Genetically Modified Bovine Herpesvirus Type 1 (BHV-1) for use to Treat Cancer

(71) Applicant: McMaster University, Hamilton (CA)

(72) Inventors: Karen Mossman, Ancaster (CA); Maria Davola, Hamilton (CA); Susan Collins, Dundas (CA); Breanne Cuddington, Kitchener (CA)

(21) Appl. No.: 18/001,828

(22) PCT Filed: Jul. 6, 2021
(86) PCT No.: PCT/CA2021/050915

§ 371 Date: Dec. 14, 2022

Related U.S. Application Data
(60) Provisional application No. 63/050,260, filed on Jul. 10, 2020.

(51) Int. Cl. A61K 35/763; C07K 14/535; A61K 45/06; C12N 7/00

(52) U.S. Cl. CPC A61K 35/763; C07K 14/535; A61K 45/06; C12N 7/00; C12N 2710/16732; C12N 2710/16722; C12N 2710/16762; C12N 2710/16771

(57) ABSTRACT

A recombinant BHV-1 oncolytic virus is provided comprising a BHV-1 mutant with enhanced cancer selectivity and/or enhance immunostimulatory activity as compared to wildtype BHV-1. The BHV-1 mutant is genetically modified to express one or more immunomodulatory molecules that induce an anti-tumor immune response. A method of generating the recombinant BHV-1 oncolytic virus is also provided.

### FIELD OF INVENTION

The present invention generally relates to oncolytic viruses, and more particularly relates to genetically modified oncolytic bovine herpesvirus type 1 (BHV-1) which targets tumor cells and induces an anti-tumor immune response.

### BACKGROUND OF THE INVENTION

Oncolytic viruses are being actively studied as novel cancer therapeutics. Oncolytic viruses preferentially target and kill cancer cells. Some viruses are naturally oncotropic while others are engineered to replicate in cancer cells. To be successful as a cancer therapeutic, an oncolytic virus must be safe and effective. Some of the first oncolytic viruses to be tested in clinical trials were derivatives of the human herpesvirus, Herpes simplex virus type 1 (HSV-1). Researchers deleted specific HSV-1 genes to promote replication within actively dividing (i.e. cancer) cells but not in normal cells. The first and only oncolytic virus that has been FDA-approved is an HSV-1 based vector called T-Vec. The use of T-Vec is limited to accessible tumors due to pre-existing immunity within the general population (50-90% of the population is latently infected with HSV-1 and thus makes neutralizing antibodies against the virus).

Bovine herpesvirus type 1 (BHV-1), a close relative of HSV-1, has exciting properties for clinical development as an oncolytic virus. BHV-1 is capable of killing immortalized and transformed cells, suggesting pre-neoplastic to overt cancer cell activity, respectively, and targeting bulk and cancer-initiating tumor cells, regardless of tumor status or subtype. Moreover, BHV-1 does not cause disease in humans. As such, there is no pre-existing immunity against this virus within the human population. When oncolytic HSV-1 is mixed with commercially available pooled human serum, the virus is neutralized and cannot infect susceptible human tumor cells. Neutralization is not seen with BHV-1, indicating a lack of neutralizing antibodies against BHV-1 in human serum.

BHV-1 glycoproteins I and E (gI and gE) are non-essential proteins that form a non-covalent-linked heterodimer in infected cells and in the virion envelope. Mutations in gE and gI do not affect cell penetration- or cell egress-kinetics of BHV-1 in vitro, but significantly decrease the size of BHV-1 plaques. BHV-1 gE and gI deletion mutants fail to form plaques in the presence of anti-BHV-1 antibodies, showing that both gE and gI glycoproteins are implicated in cell-to-cell spread mechanisms. Although BHV-1 genes encoding glycoproteins gE and gI are not essential for replication of the virus in cell culture, BHV-1 harbouring single mutations in gI or gE or double mutation in gI and gE have a strongly reduced virulence in cattle.

Since BHV-1 has all of the features required for a successful oncolytic virus, including: safety (cannot replicate in normal human cells), efficacy (replication in a wide range of cancer cell types), and wide applicability (can be injected systemically due to the lack of pre-existing immunity), it would be desirable to provide an engineered BHV-1 designed to provide effective in vivo activity.

### SUMMARY OF THE INVENTION

Genetically modified BHV-1 vectors have now been prepared which provide enhanced selectivity to target tumor cells, while secreting molecules that enhance the host anti-tumor immune response.

Accordingly, in one aspect of the invention, a recombinant BHV-1 oncolytic virus is provided comprising a BHV-1 mutant which is genetically modified to express one or more immunomodulatory molecules that induce an anti-tumor immune response. The BHV-1 mutant comprises one or more viral target endogenous genes which are at least partially deleted or altered to yield a mutant that exhibits enhanced cancer selectivity and/or enhanced immunostimulatory activity in comparison to wild type BHV-1.

In another aspect, a method of generating a recombinant BHV-1 oncolytic virus is provided comprising the steps of: i) nucleofecting a cell-line with a vector expressibly incorporating a gene that encodes an immunomodulatory molecule that induces an anti-tumor immune response, wherein said gene is incorporated within a BHV-1 target gene; and ii) infecting the cell-line with wild type BHV-1 under suitable conditions to yield a recombinant BHV-1 oncolytic virus in which the target gene is at least partially deleted or altered and the immunomodulatory molecule is expressed, and the BHV-1 oncolytic virus exhibits enhanced cancer selectivity and/or enhanced immunostimulatory activity in comparison to wild type BHV-1.

In another aspect, a method of treating an individual with cancer is provided comprising administering to the individual a BHV-1 mutant in which expression of a BHV-1 target gene is partially or fully deleted or altered to yield a mutant that exhibits enhanced cancer selectivity and/or enhanced immunostimulatory activity in comparison to wild type BHV-1.

These and other aspects of the invention will be described by reference to the following figures.

### BRIEF DESCRIPTION OF THE FIGURES

Embodiments of the invention are shown in more depth by the following descriptions to their respective drawings listed below.

FIG. 1 is a schematic of a donor plasmid that was used to create recombinant BHV-1 ΔgE mutants expressing an immunomodulatory molecule (gene of interest, GOI);

FIG. 2 graphically illustrates the production of the protein encoded by the gene of interest (human GMCSF) in the supernatant of CRIB cells infected with BHV-1 wild type (wt) or BHV-1 ΔgE-EGFP-huGMCSF isolate 3E, or nucleofected with donor plasmid from FIG. 1.;

FIG. 3 illustrates the effect of BHV-1 wt and mutants ΔgI and ΔgE on normal human lung fibroblasts (HEL) and human lung cancer cells (A549) at different multiplicities of infection (MOI, plaque forming units [pfu] per cell) as indicated by cell monolayer clearing after Giemsa staining;

FIG. 4 graphically illustrates the results from pre-clinical studies of BHV-1 ΔgI-GFP in a syngeneic murine model of melanoma, including: A) a graph illustrating tumor progression of each mouse bearing a C10 tumor treated with different combinations of mitomycin c (Mito), BHV-1 ΔgI-GFP and checkpoint inhibitors (CP); B) a graph illustrating the survival curve; C) the results of immunogenic cell death (ICD) assay; D) a graph illustrating in vitro virus replication in C10 cells in the presence or absence of Mito; and E) a graph illustrating average tumor progression of groups of mice bearing a C10 tumor treated with PBS, mitomycin c+BHV-1 ΔgI-GFP injected intratumorally+checkpoint inhibitors (Triple comb IT), or treated with mitomycin c+BHV-1 ΔgI-GFP injected intravenously+checkpoint inhibitors (Triple comb IV) with a corresponding survival curve.

FIG. 5 displays the DNA (A) and protein (B) sequence alignments of the UL49.5 gene from BHV-1 wild type (WT), BHV-1-UL49.5 Δ30-32ΔCT isolate 5C1 and the donor plasmid used to create 5C1;

FIG. 6 illustrates the production of TAP-1 in A549 cells treated with or without IFNγ (+IFNγ and −IFNγ, respectively) and infected with BHV-1 wildtype (+IFNγ+wt) or BHV-1-UL49.5 Δ30-32ΔCT isolate, 5C1 (+IFNγ+5C1), determined by Western Blot;

FIG. 7 graphically illustrates the virus replication of BHV-1 wt and UL49.5 Δ30-32ΔCT isolate 5C1 in human normal cells (HEL) and tumor cells (A549);

FIG. 8 graphically illustrates that treatment of mice bearing C10 tumors with a combination of a low dose of mitomycin c, anti-PD-L1 and anti-CTLA-4 antibodies and BHV-1 wt (Comb. with wt) or BHV-1 isolate 5C1 (Comb. with 5C1) resulted in increased tumor regression and survival;

FIG. 9 graphically illustrates the production of the protein encoded by the gene of interest (human GMCSF) in the supernatant of multiple cell types infected with the double recombinant BHV-1 mutant (UL49.5 and gE mutant) created by co-infection with BHV-1 UL49.5 Δ30-32ΔCT-RFP isolate 5C1 and BHV-1 ΔgE-EGFP-huGMCSF isolate 3E;

FIG. 10 illustrates the effect of BHV-1 wt and mutants ΔgI, ΔgE and ΔgIΔgE on normal human lung fibroblasts (HEL) and murine tumor cells (C10) at different MOI as indicated by cell monolayer clearing after Giemsa staining (A) or cell viability quantified using Alamar Blue (B); and

FIG. 11 graphically illustrates the results from pre-clinical studies of BHV-1 ΔgIΔgE in a syngeneic murine model of melanoma, including: A) a graph illustrating the survival curve of mice bearing a C10 tumor treated with triple combination of mitomycin c, oncolytic virus (BHV-1 wt, dgI, dgE or dgIdgE) and checkpoint inhibitors; and B) a graph illustrating tumor progression of each mouse.

### DETAILED DESCRIPTION OF THE INVENTION

A recombinant BHV-1 oncolytic virus is provided comprising a BHV-1 mutant with enhanced cancer selectivity and/or enhanced immunostimulatory activity in comparison to wild type BHV-1, which is genetically modified to express one or more molecules that induce an anti-tumor immune response.

Bovine herpesvirus type 1 (BHV-1 or BoHv-1) is a virus of the family Herpesviridae and the subfamily Alphaherpesvirinae, known to cause several diseases worldwide in cattle, including rhinotracheitis, vaginitis, balanoposthitis, abortion, conjunctivitis, and enteritis. The genome of BHV-1 strains comprises double-stranded DNA of about 135 kb with about 72 coding regions.

As used herein, “BHV-1 mutant” refers to a mutant virus prepared from a BHV-1 wildtype virus which is mutated to exhibit enhanced cancer selectivity and/or enhanced immunostimulatory activity. The term “enhanced cancer selectivity” is used herein to refer to a BHV-1 mutant with reduced killing capacity in normal cells, e.g. reduced killing capacity of at least about 10% as compared to the killing capacity of wildtype BHV-1, or little or no killing capacity, in normal cells such as HEL fibroblasts, and/or a BHV-1 mutant with greater killing capacity in cancer cells, e.g. a greater killing capacity of at least about 10% as compared to the killing capacity of wildtype BHV-1 in cancer cells such as A549 cells. Virus killing capacity may be visually determined as the area of cell monolayer that is cleared after virus infection and Giemsa staining. Virus killing capacity may also be determined as reduction of cell viability after a period of time following viral infection, where cell viability is quantified using commercial cell viability assays, such as, but not limited to, AlamarBlue, MTS test or CellTiter-Glo.

The term “enhanced immunostimulatory activity” is used herein to refer to a BHV-1 mutant that has lost the function of a viral protein involved in evading the host immune defence, e.g. viral UL49.5 protein that activates degradation of the transporter associated with antigen processing (TAP), to result in a stimulation of host immune defence. Loss of function of such a viral protein in a BHV-1 mutant is determined based on the expression of the cellular gene or protein involved in immune surveillance, e.g. expression of TAP. For example, a BHV-1 mutant with enhanced immunostimulatory activity is identified when it exhibits reduced or no expression of the viral protein (e.g. UL49.5 or other protein involved in evading host immune defence), or exhibits little or no affect on host cell immune defence on infection, i.e. host immune defense is essentially equivalent to that of non-infected host cells. Thus, host cells infected with a BHV-1 UL49.5 mutant will exhibit the same level of TAP expression as that of uninfected host cells. Cellular gene or protein expression is determined using methods such as, but not limited to, quantitative polymerase chain reaction (qPCR), enzyme-linked immunosorbent assay (ELISA) or Western Blot techniques.

Generally, BHV-1 mutants may be produced by deleting or altering the nucleic acid backbone of the virus at sites within one or more target genes to yield a mutant with enhanced cancer selectivity and/or enhanced immunostimulatory activity in comparison to wild type BHV-1, including deletion of an entire target gene, or deletion or alteration of one or more portions of a target gene, to block, fully or at least partially, or otherwise alter expression of, a BHV-1 gene such as a gene related to virulence of BHV-1 to yield a BHV-1 with enhanced cancer selectivity. For example, genes which affect viral replication; genes that affect host defense mechanisms; genes that affect tropism, spread throughout the body and transmissibility of the virus; and genes that encode or produce products that are directly toxic to the host are target genes which may be mutated to yield a BHV-1 mutant with enhanced cancer selectivity and/or enhanced immunostimulatory activity. Examples of BHV-1 genes that may be deleted or altered to enhance cancer selectivity include, but are not limited to, glycoprotein E gene (gE) and glycoprotein I gene (gI), and an example of a BHV-1 gene that may be altered to enhance immunostimulatory activity is the UL49.5 gene.

In one embodiment, a BHV-1 mutant is provided comprising a mutation that blocks expression of one or more glycoproteins involved in viral cell-to-cell spread. For example, the BHV-1 mutant comprises a gene mutation that abolishes expression of one or more of the glycoproteins, gI and gE. As one of skill in the art will appreciate, various mutations may be incorporated within the BHV-1 nucleic acid backbone to yield such mutants, including altering the target glycoprotein gene or deleting the entire glycoprotein gene.

In another embodiment, a BHV-1 mutant comprising mutations which allow antigen presentation to result in stimulation of a host immune response are provided. In one example, a mutant is provided which expresses mutant UL49.5 that does not degrade the host immune defence proteins, TAP1 or TAP2. The UL49.5 protein interferes with peptide translocation by inhibition of the transporter associated with antigen processing (TAP). Examples of UL49.5 mutants include, but are not limited to, mutants in which amino acids 30-32 (RRE) and the C-terminal tail (via introduction of a stop codon) are deleted.

In a further embodiment, BHV-1 mutants comprising at least two mutations are provided, for example, mutations in different BHV-1 target genes. Thus, a BHV-1 mutant in which a glycoprotein involved in viral spread is blocked as well as blockage or inactivation of a protein to result in decreased viral replication in healthy cells or a mutation which results in an increased host immune response. In one example, a mutant is provided in which at least one of gI and gE expression is blocked and in which UL49.5 is inactivated. In another example, a mutant is provided in which both gI and gE expression is blocked, and UL49.5 may optionally additionally be inactivated.

The selected BHV-1 mutant virus with enhanced cancer selectivity and/or enhanced immunostimulatory activity may be further genetically modified to express one or more immunomodulatory molecules that potentiate the virus-mediated anti-tumor immune response.

This modification may be made using recombinant technology to insert a construct into the viral genome that is adapted to express a gene of interest, for example, a gene encoding an immunomodulatory molecule of interest. Examples of immunomodulatory molecules that induce an anti-tumor response include, but are not limited to, immunomodulatory molecules such as: ecto-CRT (ecto-calreticulin), HMGB1 (high mobility group box 1 protein), chemokines and cytokines to increase the immunogenicity of the tumors including interferons such as interferon-alfa (INF-α), interleukins such as interleukin-2 (IL-2), and hematopoietic growth factors such as erythropoietin, IL-11, granulocyte-macrophage colony-stimulating factor (GM-CSF) and granulocyte colony-stimulating factor (G-CSF).

Generally, the construct comprises a gene of interest with restriction enzyme sites at both ends thereof to facilitate insertion of the gene of interest into a platform vector (such as a plasmid) adjacent to and downstream of a promoter to drive expression of the gene of interest and within flanking regions homologous to a viral sequence. In one embodiment, the homologous viral sequence in the platform vector comprises the gE locus of BHV-1 engineered to receive the construct within this locus. The BHV-1 gE protein is a non-essential protein, thus is not required for virus replication, but facilitates neuronal spread of the virus. In another embodiment, the homologous viral region in the platform vector comprises the gI locus of BHV-1 engineered to receive the construct within this locus.

The promoter within the construct may be any promoter suitable to drive expression of the gene of interest. Commonly used promoters include the CMV, PGK1, EF1a, SV40 and CAG promoters. The platform vector may also include a reporter gene that is not natively expressed in the cell to be used to confirm incorporation of the vector into BHV-1. Commonly used reporter genes include those that express a visually identifiable result, e.g. that express fluorescent or luminescent proteins, such as green fluorescent protein (GFP or EGFP), red fluorescent protein (RFP) or blue fluorescent protein (BFP), or an enzyme that catalyzes a reaction that generates light/colour, e.g. the enzyme luciferase which catalyzes a reaction with luciferin to produce light.

Recombinant BHV-1 mutant is generated by introduction of the platform vector encoding the gene of interest into the wildtype BHV-1 genome. Generally, recombinant BHV-1 mutant is generated by co-transfection of wildtype BHV-1 genomic DNA and the platform vector within a cell-line that permits replication of BHV-1 and supports efficient transfection of BHV-1 with the platform vector. Transfection may be achieved by chemical methods, for example, using calcium phosphate, cationic polymers or liposomes, or by mechanical methods such as electroporation, sonoporation or optical transfection, or using particle methods, e.g. particle bombardment or delivery via a gene gun.

In a preferred embodiment, transfection of a selected cell line with the platform vector is conducted using a modified electroporation technique known as ‘nucleofection’ which uses a combination of electrical parameters, generated by a device called a Nucleofector, with cell-type specific reagents. The vector is transferred into the cell nucleus and the cytoplasm. Optimal nucleofection conditions depend upon the individual selected cell type, not on the vector being transfected, and a number of nucleofection programs and reagent kits are available for use. In one embodiment, nucleofector kit R and program X-100 are used to transfer the platform vector into selected cells, e.g. CRIB cells (bovine cells resistant to BVDV infection), for uptake by BHV-1 to form a BHV-1 mutant. Other nucleofection kits and programs may also be used.

Following nucleofection, the cells are infected with wildtype BHV-1 using techniques well-established in the art.

Successful generation of the recombinant BHV-1 mutant may be determined based on the expression of the reporter gene or the gene of interest by the recombinant BHV-1 mutant. To facilitate detection and isolation of the recombinant BHV-1 mutant, an enrichment step may be used to isolate cells expressing the reporter gene. For example, fluorescence assisted cell sorting (FACS) may be utilized.

A BHV-1 mutant, either genetically modified to express one or more immunomodulatory molecules or not, may be administered to an individual in the treatment of a cancer, including but not limited to, lung, colon, breast, prostate, renal, ovarian, CNS cancers, etc. Generally, the present BHV-1 mutant may be formulated for administration in a pharmaceutically acceptable carrier that is not unacceptably toxic or otherwise unsuitable for administration to an individual, while not adversely affecting the viability and activity of the BHV-1 mutant. As one of skill in the art will appreciate, the selected carrier will vary with intended mode of administration of the formulation. In one embodiment, the BHV-1 mutant is formulated for administration by infusion or injection, e.g. subcutaneously, intraperitoneally, intramuscularly, intravenously or intratumorally, and thus, is formulated as a suspension in a medical-grade, physiologically acceptable carrier, such as an aqueous solution in sterile and pyrogen-free form, optionally, buffered or made isotonic. The carrier may be distilled water, a sterile carbohydrate-containing solution (e.g. sucrose or dextrose) or a sterile saline solution comprising sodium chloride and optionally buffered. Suitable sterile saline solutions may include varying concentrations of sodium chloride. Saline solutions may optionally include additional components, e.g. carbohydrates such as dextrose, sucrose, and the like. Examples of saline solutions including additional components, include Ringer's solution, e.g. lactated or acetated Ringer's solution, phosphate buffered saline (PBS), TRIS ((hydroxymethyl)aminomethane)-buffered saline (TBS), Hank's balanced salt solution (HBSS), Earle's balanced solution (EBSS), standard saline citrate (SSC), HEPES-buffered saline (HBS) and Gey's balanced salt solution (GBSS). The formulation may also include cryoprotectants.

The BHV-1 mutant may be administered in a therapeutically effective amount to an individual for the treatment of cancer. The term “individual” is used herein to refer to human and non-human mammals (e.g. excluding cattle), and preferably, to humans. The term “therapeutically effective amount” is an amount of BHV-1 mutant sufficient to treat cancer, while not exceeding an amount which may cause significant adverse effects. As one of skill in the art will appreciate, dosages of BHV-1 mutant that are therapeutically effective will vary on many factors including the nature of the condition to be treated, the individual being treated, and the mode of administration. Suitable dosages may be determined using appropriately controlled trials. Generally, dosages in the range of about 10⁶-10¹¹ plaque forming units (pfu) of virus may be suitable, including 10⁷, 10⁸, 10⁹, and 10¹⁰ pfus.

The recombinant BHV-1 mutant virus may be utilized in combination with other anti-cancer therapies such as, but not limited to, chemotherapeutic agents, e.g. mitomycin c, dacarbazine, 5-fluorouracil, epirubicin, cyclophosphamide or 5-azacytidine, immune checkpoint inhibitors, e.g. pembrolizumab or nivolumab; immunogenic antibodies, or immune cell therapy such as tumor-infiltrating lymphocytes (or TILs) or chimeric antigen receptor (CAR) T-cell therapy.

The present recombinant BHV-1 mutant advantageously provides an oncolytic virus that exhibits enhanced killing capacity in a wide range of cancer cell types as compared to normal human cells and/or enhanced immunostimulatory activity, while additionally expressing an immunomodulatory molecule that enhances its anti-tumor effect. The BHV-1 is similarly efficacious administered intravenously or intratumorally. In addition, a method of generating the present recombinant BHV-1 mutant is also provided which overcomes obstacles in its generation.

Embodiments of the invention are described in the following specific example which is not to be construed as limiting.

### Example 1—Generation of BHV Recombinant Mutants

BHV recombinant mutant useful as an oncolytic virus was prepared and genetically modified to express a gene of interest. Methods and materials are described below.

The following cells and viruses were used:
1) CRIB cells obtained from Dr. Clinton Jones (University of Oklahoma). These are a derivative of MDBK cells that are resistant to BVDV infection. BHV-1 was grown in CRIB cells and had a titre of 2.6×10⁸ pfu/mL, demonstrating that CRIB are similar to MDBK in terms of BHV-1 growth.
2) U2OS—obtained from ATCC
3) HEK 293—obtained from Dr. Frank Graham
4) BHV-1 Cooper strain—Wildtype BHV-1 was obtained from the ATCC (catalog number VR-864, lot 61236466).

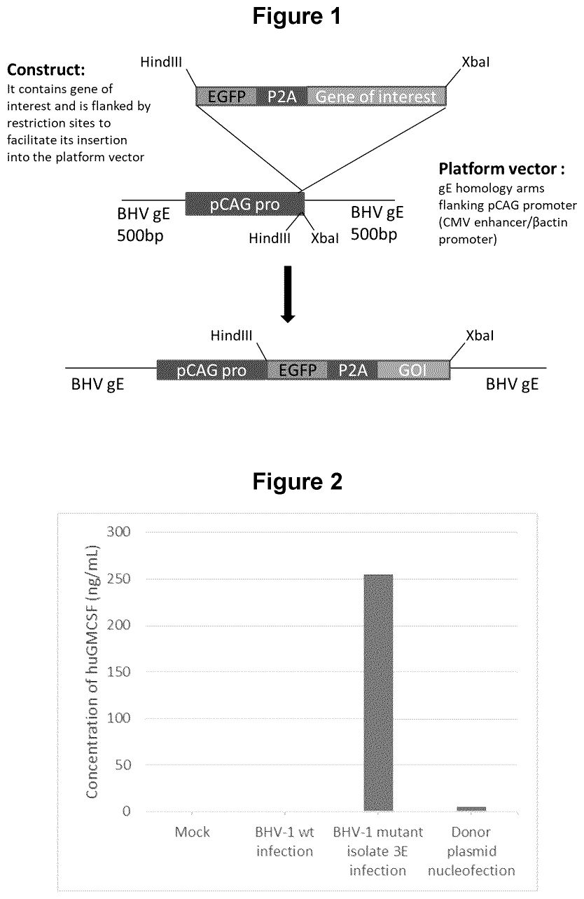

Co-transfection of viral DNA+donor plasmid—Attempts to use co-transfection of viral DNA (BHV-1 wildtype) and a donor plasmid (as shown in FIG. 1) were unsuccessful, due to the difficulty finding a cell line that is both transfected and permissive for viral replication. We attempted recombination with the co-transfection method in HEK-293 cells and CRIB cells, but failed to recover any virus.

Transfection-Infection—Since no virus was recovered following co-transfection of viral DNA, an alternate strategy was employed. The transfection-infection method consisted of transfecting the donor plasmid using lipid-based reagent (as in FIG. 1) into various cell lines, and subsequently infecting the cells with virus (BHV-1 wild type). This was minimally successful in various cell lines as shown below (Table 1).

TABLE 1
Cell lines tested for Transfection-Infection

| Cell line | Transfection efficiency | Viral replication | Success |
|---|---|---|---|
| U2OS | 20% | Low | no |
| HEK-293 | 80% | Very low | no |
| CRIB | 2% | High | no |

In order to obtain recombinant virus, a cell line that can be efficiently transfected with the donor plasmid, and also support high BHV-1 replication, is required. Unfortunately, this method did not result in the isolation of recombinant virus.

Nucleofection-Infection—The low efficiency of transfection of MDBK cells (the parental cell line for CRIB cells) with lipid-based transfection reagents has been reported. Since CRIB cells support efficient viral replication, alternate methods of delivering plasmid DNA to these cells were explored, e.g. nucleofection. Nucleofection (such as the Amaxa Nucleofector™ technology) uses a combination of cell-specific reagents and electrical parameters to open pores in both the plasma and nuclear membranes of the cell. This technology has been reported as a method of DNA transfer into cell lines that are difficult to transfect (Hamm et al. Tissue Eng. 2002; 8(2):235-45; Maasho et al. J Immunol Methods. 2004; 284(1-2):133-40). An additional advantage of this technology for the protocol is that it allows delivery of the DNA directly into the nucleus, placing it in the same location where viral replication occurs.

CRIB cells (1×10⁶) were combined with 1.5 ug pMAX-GFP plasmid DNA (nucleofection control plasmid) and nucleofected using the Amaxa Nucleofector™ II instrument, Nucleofector™ kit R and program X-001. Pictures were taken using an inverted fluorescence microscope, 5 hours post-nucleofection. Approximately 75% of cells showed GFP fluorescence, indicating uptake of the plasmid DNA. With this improvement in the ability to deliver DNA to CRIB cells, generation of recombinant virus by either co-nucleofection (of viral DNA+donor plasmid) or the nucleofection-infection protocol was conducted as described below.

Generation of BHV-1 ΔgE-EGFP-huGMCSF—The platform plasmid was ordered from Genscript (cloneID:X31749) with a pCAG promoter (CMV enhancer/chicken β-actin promoter). A construct was prepared comprising a gene of interest for insertion into the gE locus of BHV-1 within the platform plasmid. The construct contained green fluorescent protein (EGFP) and a gene of interest (GOI) (human GMCSF) separated by a P2A self-cleavage (ribosomal skip) site. This construct was cloned into the platform plasmid downstream of the promoter and within flanking BHV-1 gE sequence, as shown in FIG. 1, to facilitate homologous recombination. Recombinants are screened by the presence of EGFP. The P2A site cleaves EGFP protein from the protein expressed by the gene of interest. huGMCSF gene is included in T-VEC genomic sequence and can be measured by ELISA in the cell supernatant. Following plasmid delivery to CRIB cells, we were able to detect the expression of GMCSF in the cell culture media, by ELISA. The nucleofection-infection protocol was followed, and we screened for recombinant virus by fluorescence microscopy. In eight 6-well plates that were screened, one fluorescent plaque was observed.

To facilitate the detection and isolation of recombinant virus, we added an enrichment step, which used fluorescence assisted cell sorting (FACS) to isolate CRIB cells expressing EGFP following viral infection (approximately 0.05% of the cell population). Following FACS enrichment, the EGFP positive cells were plated onto a naïve monolayer of CRIB cells to look for viral plaques expressing EGFP. A total of eight GFP positive plaques were obtained in two 6-well plates and several of these were purified. The isolate 3E was confirmed by sequencing and expression of human GMCSF was also confirmed by ELISA (FIG. 2). No GMCSF was detected following infection with wild-type virus (BHV-1 wt). GMCSF production by nucleofection of cells with donor plasmid (from FIG. 1) was significantly lower than by infection with BHV-1 isolate 3E.

Validation of BHV-1 Recombination/Purification Strategy—A plasmid containing the EGFP-P2A-muGMCSF sequence was obtained from Genscript (cloneID: M17993) to be cloned into the ΔgE platform vector. Nucleofection-infection and FACS purification strategies were used to make the BHV-1-ΔgE-EGFP-murineGMCSF virus (virus isolate A5). The sequence of this virus has been confirmed by sequencing. Expression of murine GMCSF in the cell culture medium has been confirmed by ELISA.

### Protocols

BHV-1 Recombination Schedule

Day 1—design sequence

Day 24—receive plasmid DNA

Day 24—nucleofect/infect CRIB

Day 26—harvest virus & titre

Day 29—stain titre plates

Day 31—split CRIB into T150 for sorting

Day 32—infect T150 CRIB
Prepare for cell sorting at 12-18 hr post-infection
Plate the sorted cells into 96-well plates at a concentration of 0.3 cells/well with uninfected CRIB ˜60% confluent

Day 34—Screen for GFP+ cells in the 96-well plate by Typhoon & confirm by fluorescence microscopy

Day 36—Harvest virus from GFP+ cells

Day 39—Dilute the virus & infect a 96-well plate of CRIB cells at 0.3 pfu/well to get isolated virus particles

Day 43—Repeat dilution in 96-well plate of CRIB at 0.3 pfu/well

Day 46—Titre virus to ensure that all plaques express GFP. If any GFP-negative plaques are found, further purification is required.

Day 48—if virus is plaque pure—infect 1 T150 for seed stock

Day 51—harvest prep & titre

Day 54—infect 20 x T150 for full prep

Day 56—harvest

Day 57—purification

Day 58—titre

Day 61—ready for characterization

BHV-1 Nucleofection/Infection Protocol for Homologous Recombination

Nucleofection:

1. Take nucleofector reagents (Kit R) out of 4° C. & allow them to warm to room temperature

2. Add 2 mL DMEM+5%HS to each well of a 6-well plate & equilibrate in 37° C. incubator

3. Trypsinize CRIB cells (1xT150)

4. Transfer 1×106 cells per reaction to a 15 mL conical tube

5. Spin at 650 rpm, 10 min, room temp

6. Resuspend cell pellet in 100 ul (per reaction) Nucleofector solution R (82 ul nucleofector solution+18 ul supplement)

7. Prepare sterile Eppendorf tube with plasmid DNA (1-2 ug)

8. Transfer 100 ul cells (in nucleofector solution) to the Eppendorf tube containing DNA

9. Transfer cells/DNA to cuvette

10. Nucleofect with program X-001

11. Add 500 ul pre-equilibrated media to cuvette

12. Transfer cells to 1 well of 6-well containing 1.5 ml pre-equilibrated media

13. Return plate to incubator

Infection

1. Check fluorescence on microscope

2. At 5 hr post-nucleofection, infect cells with BHV (MOI=0.05)
Wash cells with PBS
Infect with 400 ul per well, 1 hr, 37° C., rocking every 10 minutes
Add back DMEM+1%FBS

Harvest Virus

1. Harvest when 100% CPE is observed (usually the next day)

2. Pipette to detach cells into media

3. Transfer to 15 mL conical tube

4. Freeze (−80° C.)/Thaw(37° C.) 3X

5. Sonicate 1×30 sec

6. Spin at 1500 rpm, 10 min, 4° C.

7. Transfer supernatant to 2 mL tube

8. Keep 25 ul aliquot to titre & freeze remaining virus at −80° C.

Flow-Sorting for Recombinant BHV Enrichment

1. Seed 1xT150 for each recombination reaction to be screened (+1 T150 for -ve control)

2. The next day, infect at MOI=0.1 (for nucleofection/infection) or MOI=0.01 (for co-infection)

3. At the time of infection, prepare the flow sorting tubes (4 mL, polypropylene) by filling with collecting buffer (media+1%L-glutamine+30%FBS)

4. At 12 hours post-infection, prepare samples as follows:

5. Wash cells with PBS

6. Add 2 mL TrypLE

7. Return to 37 degree incubator until cells begin to detach

8. Add 10 mL DMEM+5%FBS to inactivate TrypLE

9. Spin down at 650 xg, 10 min, 4 degrees

10. Resuspend pellet in sorting buffer (PBS, 1%BSA, 5 mM EDTA)

11. Count cells with heamocytometer

12. Dilute to 2.5×106 cells/mL

13. Force through filter caps immediately before sorting

Cells are sorted. This is an enrichment step, rather than purification. The sorted cells are added to a naive CRIB monolayer in 96-well plates, plated at 0.3 sorted cells/well

After sorting:

1. Determine the total number of cells sorted into the GFP+ve population

2. Add 0.3 sorted cells/well of 96-well plate containing a CRIB monolayer (˜60% confluent)

3. Leave these plates in the incubator overnight

4. Monitor for GFP expression

5. When plaques are observed, screen for GFP+ve plaques on the fluorescence microscope

6. Harvest all of the cells & virus from the GFP+ve well.

7. Repeat dilution & isolation in 96-well plates until the virus is pure.

### Example 2—BHV-1 ΔgE and ΔgI mutants

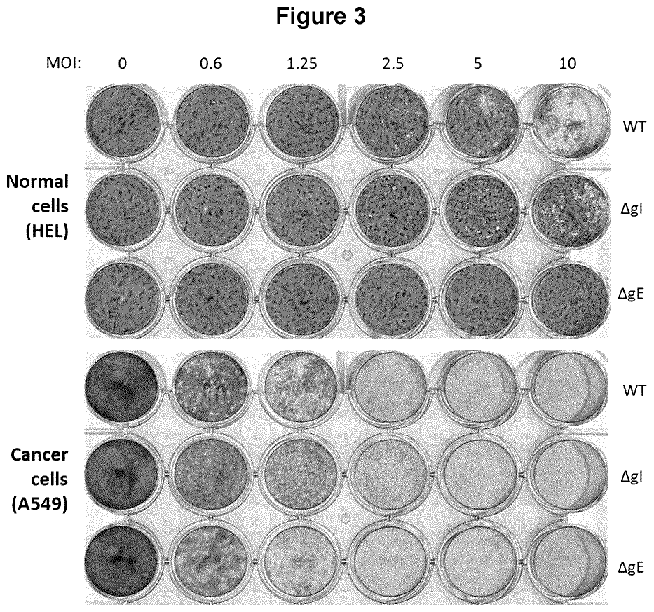

BHV-1 ΔgE mutants were created as shown in Example 1. BHV-1 ΔgE-EGFP-muGMCSF mutant was compared with a BHV-1 ΔgI-GFP mutant which was obtained from Dr. Günther Keil (Friedrich-Loeffler-Institut, Germany). For in vitro characterization of their safety profile, human lung fibroblasts (HEL) and adenocarcinoma cells (A549) were infected with different MOIs of BHV-1 wt and gE and gI deletion mutants (ΔgE and ΔgI). Both BHV-1 deletion mutants showed lower killing capacity in normal human fibroblasts at the maximum tested MOI compared to the wild type, the ΔgE mutant being the least cytopathic (FIG. 3). The wild type and dgE mutant showed similar killing capacity in A549 cancer cells. Both mutants have similar killing capacity in C10 murine tumor cells. These results show that both mutants have a greater cancer selectivity than wildtype suggesting that both deletions may result in a virus with a better safety profile than wild type while retaining the ability to kill tumor cells.

In addition, pre-clinical studies of BHV-1 ΔgI-GFP were performed. BHV-1 ΔgI-GFP was tested in the C10 tumor model in C57Bl/6 mice (Miller et al. Mol Ther. 2001;3(2):160-8). The C10 model is the first syngeneic murine model of cancer that is susceptible to BHV-1 entry. Murine cells are not susceptible to BHV-1 as they lack essential receptors for BHV-1 entry. C10 is a B16 mouse melanoma cell clone expressing human nectin-1. The C10 cells are able to form tumors reproducibly in C57Bl/6 mice, and human nectin-1 expression does not induce detectable in vivo immunogenicity against tumors. Preliminary in vitro results showed that nectin-1 expression significantly improves BHV-1 entry to unsusceptible B16 cells.

The following treatment regimen was used. Once tumors reached treatable size, they were treated intratumorally with one dose of 100 μg mitomycin C (day 1) and intratumorally or intravenously with 3 doses of 2×10⁷ plaque-forming units (pfu) of BHV-1 ΔgI-GFP (days 2, 3 and 4) and intraperitoneally with α-CTLA-4 and α-PD-L1 checkpoint blockade antibodies (200 μg each) from day 1 every 3 days for a total of 10 doses.

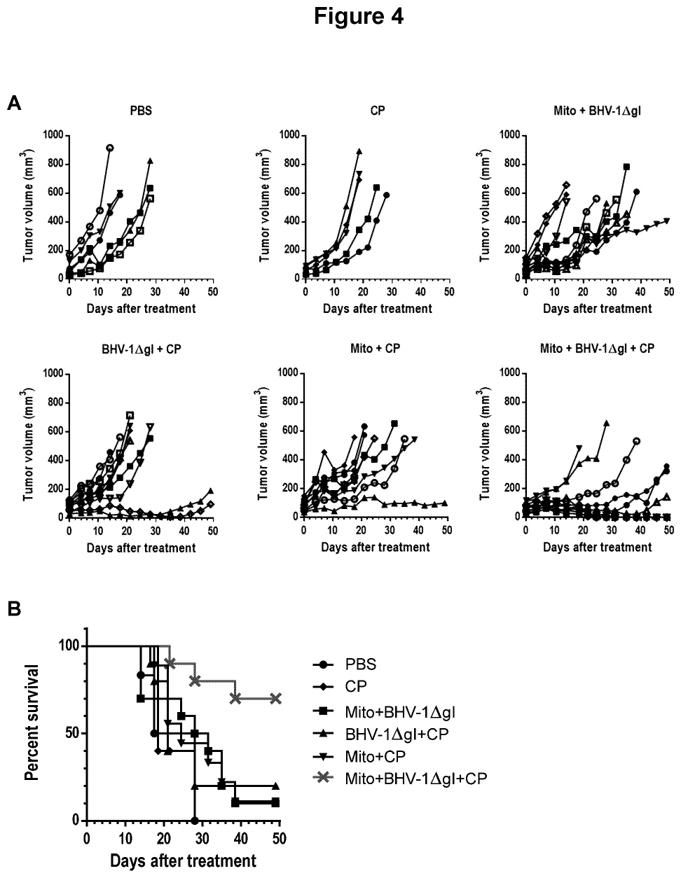

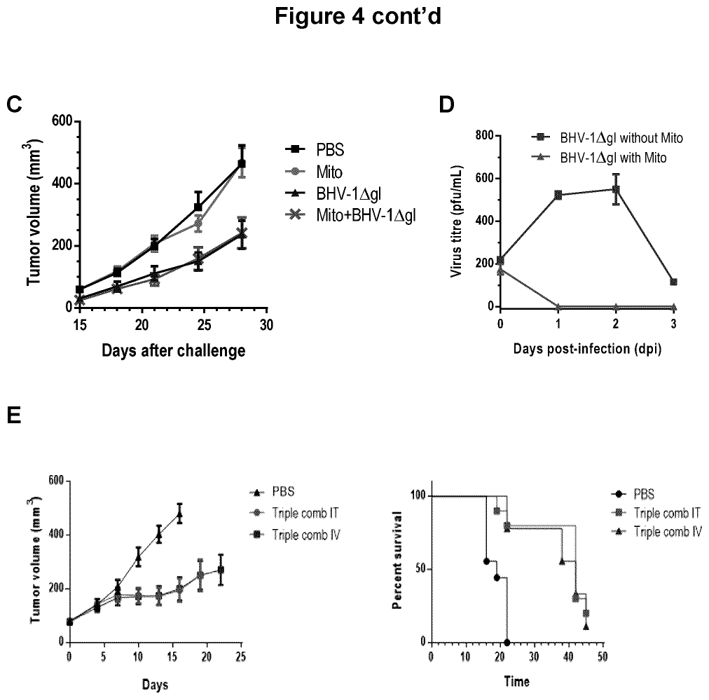

BHV-1 ΔgI-GFP showed enhanced tumor control (FIG. 4A/4E) and animal survival (FIG. 4B/4E) over the control groups, even when BHV-1 ΔgI-GFP infection was low-productive in vitro, and the addition of chemotherapy blocked totally the virus replication (FIG. 4C). In addition, BHV-1 ΔgI-GFP low-productive infection is sufficient to induce a host immune response against C10 tumors (FIG. 4D). The initiation or augmentation of a systemic antitumor immune response can be partially explained by the ability of therapies such as oncolytic viruses to induce immunogenic cell death (ICD) of cancer cells. The gold standard assay to assess ICD uses dying tumor cells as a vaccine to determine if the type of cell death is sufficient to induce an immune response capable of limiting or controlling subsequent tumor formation (Kepp et al. Oncoimmunlogy. 2014:3(9):e955691). Using this assay, we found that vaccination with dying cells infected with BHV-1 ΔgI-GFP was more effective than treatment with mitomycin c alone, and as effective as dying cells treated with mitomycin c and BHV-1 ΔgI-GFP, in limiting tumor growth (FIG. 4D). Thus, BHV-1 ΔgI-GFP is sufficient to induce ICD of C10 cells, even when de novo virus production is very low or even blocked by mitomycin c. Thus, in vivo antitumor activity does not correlate with virus replication capacity. While previous findings do show that in vivo efficacy of oncolytic viruses correlates with the ability to activate an antitumor immune response, our findings do not follow the same tendency highlighting the uniqueness of BHV-1 mechanism of action.

This work demonstrates the enhanced cancer selectivity of recombinant BHV-1 ΔgE and ΔgI mutants and the in vivo efficacy of recombinant BHV-1 ΔgI mutant when combined with low dose chemotherapy and checkpoint immunotherapy. This work also demonstrates that intravenous administration is as effective as intratumoral administration in terms of inhibition of tumor progression and survival.

### Example 3—BHV-1 UL49.5 Mutant

The BHV-1 UL49.5 gene product, glycoprotein N, normally blocks antigen presentation within virally infected cells to allow maximum virus replication and spread by “hiding” the virus from the immune system. However, the most important goal of an oncolytic virus is believed to be the ability to attract and stimulate the immune system against cancer, allowing for presentation of both viral proteins and tumor-specific proteins. Thus, a mutation in UL49.5 gene will facilitate a more robust anti-cancer immune response by increasing antigen presentation.

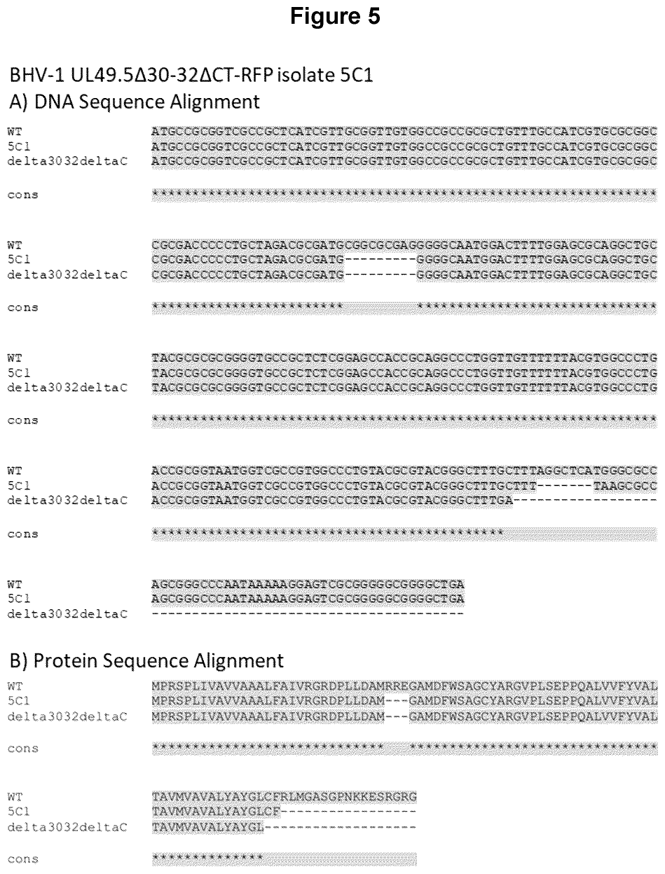

Thus, a BHV-1 mutant, BHV-1-UL49.5Δ30-32ΔCT mutant, was created to retain host transporter associated with antigen processing 1 (TAP-1) expression and function. Specifically, two mutations were engineered into the BHV-1 UL49.5 gene, the deletion of amino acids 30-32 (RRE) and the deletion of the C-terminal tail (via introduction of a stop codon). Engineered virus was plaque purified and the UL49.5 gene sequenced to validate the two mutations. Nucleic acid (FIG. 5A) and protein (FIG. 5B) sequence alignment of UL49.5 gene from wild type (wt) and recombinant virus (clone 5C1) and donor plasmid (delta3032deltaC) are shown in FIG. 5. The consensus (cons) sequence confirms the lack of additional mutations within the UL49.5 gene and corresponding protein.

Plasmid DNA—The plasmid (pUC57simple) used to generate the BHV-1 UL49.5 mutant included the gene sequence of UL49.5, with the desired changes, i.e. the sequence for amino acids 30-32 and the cytoplasmic tail were deleted. The plasmid was also modified to express mCherry (red fluorescent protein—RFP) and to include a P2A cleavage site immediately upstream of the start codon for UL49.5.

Generation of UL49.5Δ30-32ΔCT-RFP virus—The plasmid DNA was nucleofected into CRIB cells using nucleofector kit R and program X-100, followed by infection with wildtype BHV-1 (MOI=0.05) at 5 hours post-nucleofection. Following screening of plaques on 6-well plates, six RFP positive plaques were observed. The current isolate (5C1) was subjected to two further rounds of plaque purification and confirmed to be pure. The mutation was confirmed by PCR amplification and sequencing (FIG. 5).

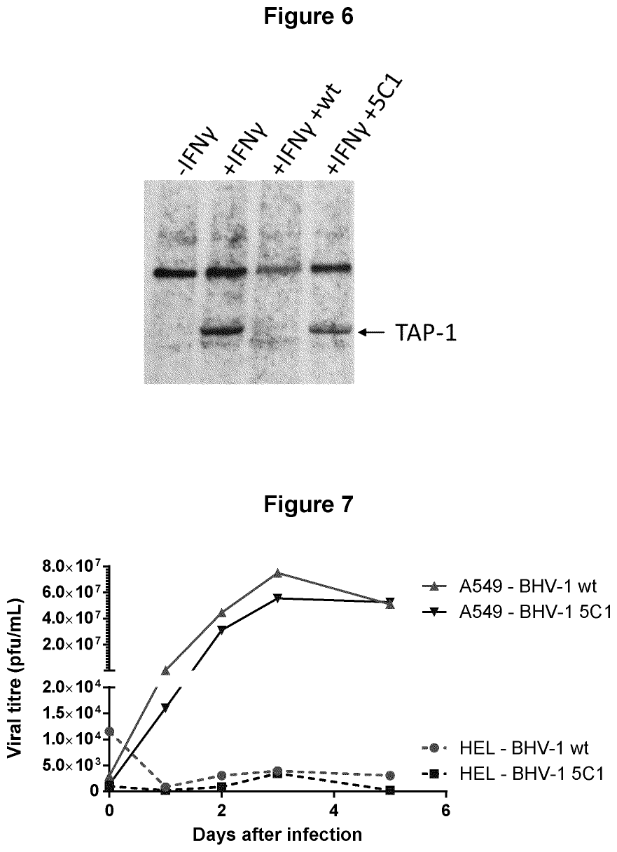

Characterization of BHV-1-UL49.5Δ30-32ΔCT—The BHV-1-UL49.5Δ30-32ΔCT isolate 5C1 was functionally confirmed to not degrade TAP-1. FIG. 6 demonstrates that TAP-1 is produced after induction with IFNγ (+IFNγ) but is degraded after further infection with BHV-1 wildtype (+IFNγ +wt). However, infecting with BHV-1 mutant 5C1 instead of wildtype fails to cause TAP-1 degradation (+IFNγ +5C1).

The BHV-1-UL49.5Δ30-32ΔCT mutant was determined to retain its therapeutic index in human cancer cells. FIG. 7 demonstrates that BHV-1-UL49.5Δ30-32ΔCT grows to similar titres as wild-type BHV-1 (wt) in human lung adenocarcinoma (A549 cells) but fails to grow in normal human lung (HEL) fibroblasts (FIG. 7). Moreover, BHV-1-UL49.5Δ30-32ΔCT induces a similar level of cytopathic effect (cell killing) as wild type over a range of multiplicities of infection. Thus, a recombinant BHV-1-UL49.5Δ30-32ΔCT mutant modified to express a selected GOI is also appropriate for use in accordance with the present invention.

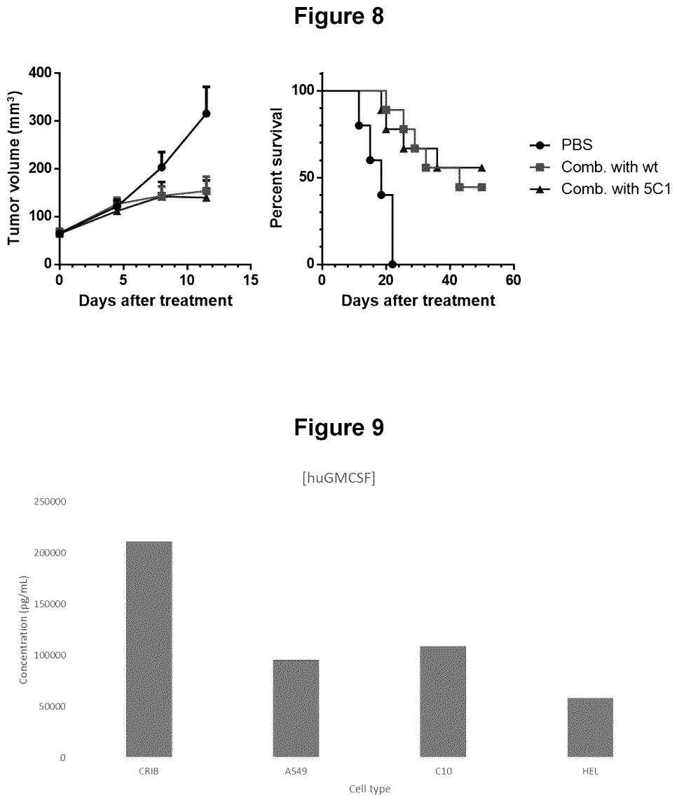

The BHV-1-UL49.5Δ30-32ΔCT isolate 5C1 was tested in the C10 tumor model, as described in Example 2, with the same treatment regimen (intratumorally with one dose of 100 μg mitomycin C (day1) and 3 doses of 2×10⁷ pfu of BHV-1 WT or 5C1 (days 2, 3 and 4) and intraperitoneally with α-CTLA-4 and α-PD-L1 checkpoint blockade antibodies (200 μg each) from day 1 every 3 days for a total of 10 doses). There was no significant difference between the WT and 5C1 BHV-1 with respect to tumor growth or survival (FIG. 8). Both triple combination with WT and 5C1 provided significant improvement compared to PBS control group with more than 2-fold increase in median survival.

This work demonstrates that BHV-1-UL49.5Δ30-32ΔCT mutant has enhanced immunostimulatory activity and retains the in vivo efficacy of the wildtype.

### Example 4—BHV-1 with Mutation in UL49.5 and Deletion in gE Expressing Human GMCSF

The vector, BHV-1-UL49.5Δ30-32ΔCT, can serve as a backbone for recombinant vectors modified by the gE plasmid construct of FIG. 1 to express GOI, including immune stimulating molecules to increase immunogenicity and induce more potent anti-tumor immune responses. Thus, the two modifications together, the viral UL49.5 mutant incorporating the gE (or gI) deletion expressing a selected GOI, allows for a more robust induction of an anti-tumor immune response than is possible with wild-type BHV-1. An additional benefit of placing molecules of interest into the gE or gI locus is that the loss of gE or gI does not interfere with replication within permissive cancer cells, but decreases the ability of the virus to kill normal healthy cells, thus increasing the safety of this vector.

Thus, a co-infection strategy was combined with FACS enrichment (as described in Example 1) to generate a double mutant virus Q5A from the previous single mutant viruses (BHV-1-UL49.5Δ30-32ΔCT isolate 5C1+BHV-1 ΔgE-EGFP-huGMCSF isolate 3E). This virus has both the mutated UL49.5 (as described in Example 3) and the gE deletion with human GMCSF expression (as described in Example 1). Expression of GMCSF from this virus was confirmed in several different cell types, even in the absence of viral replication (FIG. 9).

This work demonstrates the feasibility of generating double BHV-1 mutants, e.g. with UL49.5 mutation and gE/gI deletion and expressing an immunomodulatory molecule, e.g. GMCSF.

### Example 5—BHV-1 ΔgIΔgE Mutant

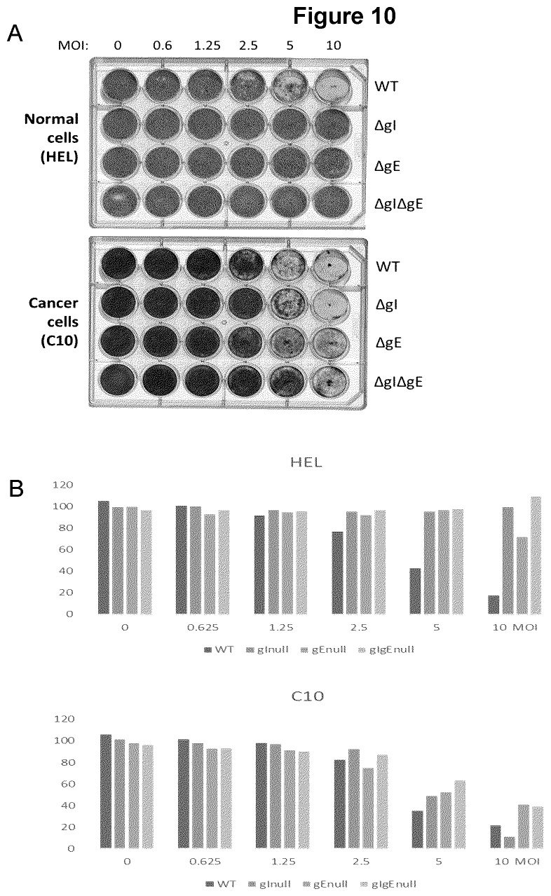

BHV-1 ΔgIΔgE mutant was created as described in Example 1. BHV-1 ΔgIΔgE mutant was compared with BHV-1 wildtype (wt) and the single mutants, BHV-1 ΔgE and BHV-1 ΔgI. For in vitro characterization of their safety profile, human lung fibroblasts (HEL) were infected with different MOIs of BHV-1 wt and mutants. All mutants showed lower killing capacity in normal human fibroblasts at the maximum tested MOI compared to the wildtype (FIG. 10A and B). The wildtype and mutants showed similar killing capacity in C10 murine tumor cells (FIG. 10A and B). These results show that BHV-1 ΔgIΔgE mutant, similar to the single mutants, has a greater cancer selectivity than wildtype indicating that either or both deletions may result in a virus with a better safety profile than wild type while retaining the ability to kill tumor cells.

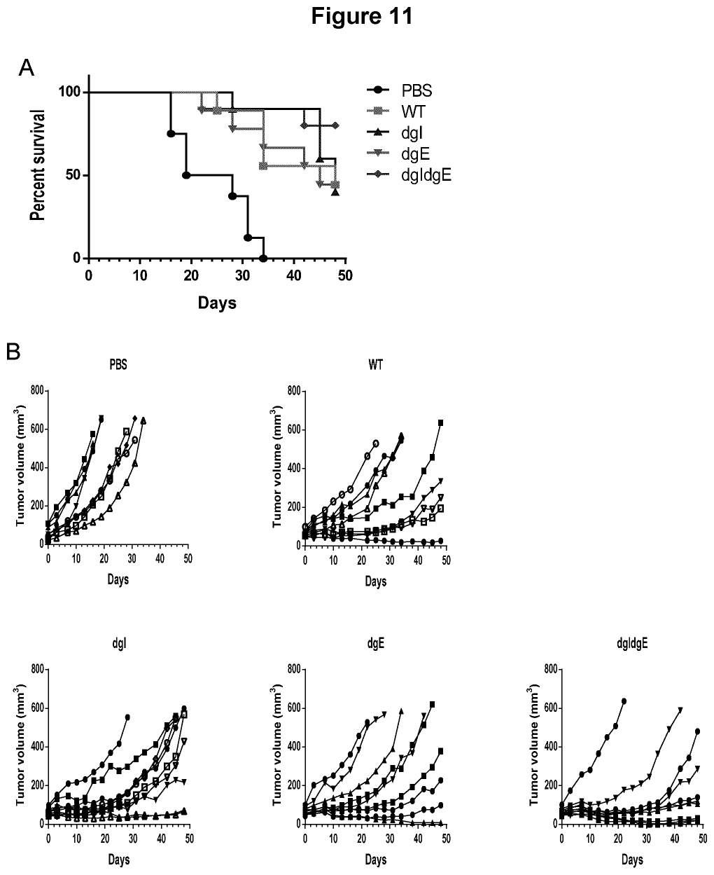

In addition, pre-clinical studies of the double BHV-1 ΔgIΔgE mutant were performed in the C10 tumor model, as described in Example 2, and compared with wild type and single mutants BHV-1 ΔgI and BHV-1 ΔgE. The same treatment regimen was used (intratumorally with one dose of 100 μg mitomycin C (day1) and 3 doses of 2×10⁷ pfu of BHV-1 WT or mutants (days 2, 3 and 4) and intraperitoneally with α-CTLA-4 and α-PD-L1 checkpoint blockade antibodies (200 μg each) from day 1 every 3 days for a total of 10 doses). All triple combinations with WT and mutants provided significant improvement of survival compared to the PBS group with more than a 2-fold increase in median survival (FIG. 11A). Moreover, the combination with double mutant BHV-1 ΔgIΔgE showed an improvement of tumor control compared to wild type (FIG. 11B).

1. A recombinant BHV-1 oncolytic virus comprising a BHV-1 mutant genetically modified to express a gene encoding an immunomodulatory molecule that induces an anti-tumor immune response, wherein one or more target genes in the BHV-1 mutant is at least partially deleted or altered to yield a mutant that exhibits enhanced cancer selectivity and/or enhanced immunostimulatory activity in comparison to wild type BHV-1.

2. The virus of claim 1, wherein the gene encoding the immunomodulatory molecule is inserted within a BHV-1 target gene.

3. The virus of claim 1, wherein the target gene expresses a glycoprotein involved in viral cell-to-cell spread.

4. The virus of claim 3, wherein the glycoprotein is gI and/or gE.

5. The virus of claim 1, wherein the BHV-1 mutant exhibits decreased killing capacity in normal human cells and/or increased killing capacity in human tumor cells as compared to the killing exhibited by wildtype BHV-1.

6. The virus of claim 1, wherein the target gene expresses a viral protein involved in host defense evasion.

7. The virus of claim 6, wherein the viral protein is UL49.5 which is mutated to prevent degradation of TAP.

8. The virus of claim 1, wherein the BHV-1 mutant comprises a mutation in at least one of gI, gE and UL49.5.

9. The virus of claim 1, wherein the immunomodulatory molecule is a chemokine or a cytokine.

10. The virus of claim 1, wherein the immunomodulatory molecule is selected from the group consisting of: ecto-CRT (ecto-calreticulin), HMGB1 (high mobility group box 1 protein), interferon, an interleukin, a hematopoietic growth factor, granulocyte-macrophage colony-stimulating factor (GM-CSF) and granulocyte colony-stimulating factor (G-CSF).

11. A method of generating a recombinant BHV-1 oncolytic virus comprising the steps of:
i) nucleofecting a cell-line with a vector expressibly incorporating a gene that encodes an immunomodulatory molecule that induces an anti-tumor immune response, wherein said gene is incorporated within a BHV-1 target gene; and
ii) infecting the cell-line with wild type BHV-1 under suitable conditions to yield a recombinant BHV-1 oncolytic virus in which the target gene is at least partially deleted or altered and the immunomodulatory molecule is expressed, and the BHV-1 oncolytic virus exhibits enhanced cancer selectivity and/or enhanced immunostimulatory activity in comparison to wild type BHV-1.

12. The method of claim 11, wherein the target gene encodes a glycoprotein involved in viral cell-to-cell spread.

13. The method of claim 12, wherein the glycoprotein is gI and/or gE.

14. A method of treating an individual with cancer comprising administering to the individual a BHV-1 mutant in which one or more BHV-1 target genes are partially or totally deleted or altered to yield a mutant that exhibits enhanced cancer selectivity and/or immunostimulatory activity in comparison to wild type BHV-1.

15. The method of claim 14, wherein the individual is a human.

16. The method of claim 14, additionally comprising administration of at least one of a chemotherapeutic agent, an immune checkpoint inhibitor or an immunogenic antibody.

17. The method of claim 14, conducted in combination with an immune cell therapy.

18. The method of claim 14, wherein the target gene expresses a glycoprotein involved in viral cell-to-cell spread.

19. The method of claim 18, wherein the glycoprotein is gI and/or gE.

20. The method of claim 14, wherein the BHV-1 mutant exhibits decreased killing capacity in normal human cells and/or increased killing capacity in human tumor cells.

21. The method of claim 14, wherein the target gene expresses a viral protein involved in host defense evasion.

22. The method of claim 21, wherein the viral protein is UL49.5 which is mutated to prevent degradation of TAP.

23. The method of claim 14, wherein the BHV-1 mutant is genetically modified to express a gene encoding an immunomodulatory molecule that induces an anti-tumor immune response.

24. The method of claim 23, wherein the gene encoding the immunomodulatory molecule is inserted within a BHV-1 target gene.

25. The method of claim 23, wherein the immunomodulatory molecule is a chemokine or a cytokine.

26. The method of claim 25, wherein the immunomodulatory molecule is selected from the group consisting of: ecto-CRT (ecto-calreticulin), HMGB1 (high mobility group box 1 protein), interferon, an interleukin, a hematopoietic growth factor, granulocyte-macrophage colony-stimulating factor (GM-CSF) and granulocyte colony-stimulating factor (G-CSF).

27. The method of claim 14, wherein the BHV-1 mutant comprises a mutation in two target genes selected from the gI, gE and UL49.5 genes.

28. The method of claim 14, wherein the BHV-1 mutant is administered intratumorally.

29. The method of claim 14, wherein the BHV-1 mutant is administered intravenously.

30. The method of claim 14, wherein the BHV-1 mutant is administered at a dosage of about 10⁶ to 10¹¹ plaque forming units.
